(19) United States
(12) Patent Application Publication
(10) Pub. No.: US 2025/0104384 A1
(43) Pub. Date: Mar. 27, 2025

(54) SYSTEM AND METHOD FOR SEGMENTATION OF MEDICAL IMAGING DATA

(71) Applicant: Siemens Healthineers AG, Forchheim (DE)

(72) Inventors: Arnaud Arindra ADIYOSO, Nuernberg (DE); Florin-Cristian GHESU, Baiersdorf (DE)

(73) Assignee: Siemens Healthineers AG, Forchheim (DE)

(21) Appl. No.: 18/888,424

(22) Filed: Sep. 18, 2024

(30) Foreign Application Priority Data
Sep. 21, 2023 (DE) 10 2023 209 205.0

(51) Int. Cl. G06V 10/26; G06F 40/279; G06F 40/30; G06V 10/77; G06V 10/82; G10L 15/22

(52) U.S. Cl. CPC G06V 10/26; G06F 40/279; G06F 40/30; G06V 10/7715; G06V 10/82; G06V 2201/032; G10L 15/22

(57) ABSTRACT

A system configured to segment medical imaging data, comprising an input unit configured to receive text data and the medical imaging data, wherein the received text data comprises a segmentation task formulated in natural language with regard to the medical imaging data; a text encoder unit configured to generate a text embedding based on the received text data; an imaging encoder unit configured to generate an image embedding based on the received medical imaging data; a segmentation unit configured to receive the generated text embedding and the generated image embedding and to determine a segmentation of the received medical imaging data via a function trained by an artificial intelligence algorithm; and an output interface configured to output the segmentation of the medical imaging data.

CROSS-REFERENCE TO RELATED APPLICATION(S)

The present application claims priority under 35 U.S.C. § 119 to German Patent Application No. 10 2023 209 205.0, filed Sep. 21, 2023, the entire contents of which are incorporated herein by reference.

### FIELD

One or more example embodiments relates to a system for providing a segmentation of medical imaging data on the basis of text input. One or more example embodiments further relates to a computer-implemented method for providing the segmentation.

Independent of the grammatical term usage, individuals with male, female or other gender identities are included within the term.

### RELATED ART

The segmentation of medical data is a fundamental method for enabling an identification of different tissue regions and lesions from an imaging method (e.g. X-ray radiation).

Apart from problems that are associated with the resolution of the image data, such as the identification of lesion borders, in particular in the case of isodense lesions, the problem often arises as to how the different criteria of different physicians fit together with the segmentation. For example, as a rule, the identification of a tissue region which contains tumors can lead, dependent upon criteria used, to different segmentations. From a computed tomography scan of the lung of a patient, the definition of a lesion (whether the solid core of a nodule, a cyst, the primary tumor of a metastasis or the whole metastasized region) is decisive in order to obtain the desired segmentation.

Conventionally, the problem is solved in that pre-defined segmentation tasks are set out in order to broaden the segmentation possibilities. In recent time, methods have been developed for using machine learning in order to carry out the method more efficiently.

### SUMMARY

However, in many cases, this conventional procedure is imprecise and fault-prone since, ultimately, pre-defined segmentation tasks have not been able, in some cases, to represent precisely the criteria of a physician.

Furthermore, this conventional approach is restricted to specific segmentation tasks. In order to be able to cover numerous application cases, a large number of segmentation methods are therefore needed, which is time-consuming and not comprehensive.

One or more example embodiments enables an improved and more adaptable segmentation of medical imaging data that is based, in particular, upon the information from text input in natural language.

This is achieved with a system having the features of claim 1 and/or with a computer-implemented method having the features of claim 13.

Preferred embodiments of the invention with advantageous features are the subject matter of the dependent claims.

### BRIEF DESCRIPTION OF THE DRAWINGS

Example embodiments will now be described making reference to the drawings. They serve further to illustrate embodiments of concepts which contain the claimed invention and to explain various principles and advantages of these embodiments.

In the drawings:

FIG. 1 shows a system for segmentation of medical imaging data according to one embodiment of the invention;

FIG. 2 shows a schematic block diagram to illustrate the sequence of a computer-implemented method for segmentation of medical imaging data according to one embodiment of the invention;

FIG. 3 shows a schematic flow diagram to illustrate the sequence of a computer-implemented method for segmentation of medical imaging data according to one embodiment of the invention;

FIG. 4 shows a schematic block diagram to illustrate the sequence of a computer-implemented method for providing a trained function according to one embodiment of the invention;

FIG. 5 shows a representation of different segmentation masks in association with the identification of lung nodules according to one embodiment of the invention;

FIG. 6 shows a schematic block diagram which represents a medical system with a medical scanning system and a system for segmentation of medical imaging data according to one embodiment of the present invention;

FIG. 7 shows a schematic block diagram which represents a computer program product according to one embodiment of the present invention; and

FIG. 8 shows a schematic block diagram which represents a non-volatile computer-readable data storage medium according to one embodiment of the present invention.

### DETAILED DESCRIPTION

According to a first aspect, one or more example embodiments provides a system for segmentation of medical imaging data which has: an input unit which is configured to receive text data and medical imaging data, wherein the input text data comprises a segmentation task formulated in natural language with regard to the medical imaging data; a text encoder unit which is configured to generate a text embedding on the basis of the received text data; an imaging encoder unit which is configured, on the basis of the received medical imaging data, to generate an image embedding; a segmentation unit which is configured to receive the generated text embedding and the generated image embedding and, via a function trained by way of an artificial intelligence algorithm, to determine a segmentation of the received medical imaging data; and an output interface which is configured to output the segmentation of the medical imaging data.

Medical imaging data comprises image data which originates from an imaging process in medical diagnostics, for example from an X-ray recording, a magnetic resonance tomography scan, a computed tomography scan, an ultrasound scan and other methods from digital pathology.

Herein, text data should be understood as meaning that a text input in natural language takes place, wherein the different segmentation tasks define a relationship with medical imaging data.

The artificial intelligence algorithm used can be an arbitrary algorithm that is based at least partially on methods of artificial intelligence.

Artificial intelligence should be understood to be a computer-aided unit or facility that is configured to implement various data analysis methods which are generally also referred to as artificial intelligence, machine learning, deep learning, computer learning or suchlike.

Herein and in the following, an artificial neural network should be understood to be a computing system which is based upon a collection of connected units or nodes structured in layers that are capable of transferring signals to one another and which preferably at least partially comprises a transformer network architecture.

A trained function should be understood as a function which is based upon an artificial neural network.

An image embedding is a representation of an image via which the image is mapped onto an image feature vector. A convolutional neural network (CNN) can be used for generating the image embedding.

A text embedding is a representation of a text via which the text is mapped onto a text feature vector. A convolutional neural network (CNN) can be used for generating the text embedding.

An embedding simplifies the processing of the information contained in the image, for example by way of modeling the machine learning.

The segmentation unit should be understood to be an image decoder which is configured to create, from the image embedding and with the aid of the text embedding, an image with the segmentation.

The different facilities and the output interface comprise, in general, devices and computer programs which can receive, process and evaluate data. The facilities and the output interface are connected to one another, by cable and/or wirelessly, to be able to exchange signals. Therefore, the facilities comprise at least one central processing unit (CPU) and/or at least one graphics processing unit (GPU) and/or at least one field-programmable gate array (FPGA) and/or at least one application-specific integrated circuit (ASIC) and/or an arbitrary combination of the aforementioned facilities. Each facility can further comprise a working memory store which is operatively connected to the at least one CPU, and/or a non-volatile memory store which is operatively connected to the at least one CPU and/or the working memory store. Each facility can be implemented partially and/or completely in a local device and/or partially and/or completely in a remote system, such as a cloud computing platform.

The various facilities and the output interface can be realized in hardware and/or software, connected by cable and/or wirelessly and in any combination thereof. They can also comprise an interface to an intranet or the internet, to a cloud computing service, to a remote server and/or suchlike.

The text encoder unit, the imaging encoder unit and the segmentation unit can execute, in particular, a software item, a program routine, an app or an algorithm with different capabilities for data processing.

The input unit and the output interface can comprise, in particular, an interface, for example an application programming interface (API), so that the input unit, in particular, can read in the text data and the imaging data and the output interface can output the processed imaging data with the segmentation.

In cloud computing-based systems, a large number of devices is connected via the internet to the cloud computing system. The devices can be situated in a remote facility that is connected to the cloud computing system. The devices can comprise or consist of, for example, devices, sensors, actuators, robots and/or machines in one or more industrial plants. The devices can be household appliances, office machines in a living/business facility or suchlike.

The cloud computing system can enable the remote configuration, monitoring, control and maintenance of the connected devices (also generally designated “assets”). Furthermore, the cloud computing system can facilitate the storage of large data quantities that are often acquired by the devices, the analysis of the large data quantities and the provision of findings (e.g. performance indicators, outliers, etc.) and warnings for operators, field technicians or owners of the devices via a graphical user interface (e.g. of web applications). The findings and warnings can enable the control and maintenance of the devices, which leads to an efficient and failure-resistant operation of the devices. The cloud computing system can also enable the amendment of parameters that are associated with the devices. Finally, the cloud computing system can also output control commands which have been obtained, for example on the basis of the findings and warnings, via the graphical user interface.

The cloud computing system can comprise a large number of servers or processors (also known as “cloud infrastructure”), which are geographically distributed and are connected to one another via a network. Installed on the servers/processors is a special platform (referred to below as “cloud computing platform”) which provides the aforementioned functions as a service (referred to below as “cloud service”). The cloud computing platform can comprise a large number of software programs that are executed on one or more servers or processors of the cloud computing system, in order to enable the provision of the required service or services for the devices and their users.

One or more APIs are utilized in the cloud computing system in order to offer various cloud services to the users.

One or more example embodiments further provides, according to a second aspect, a computer-implemented method for segmentation of medical imaging data, having the steps: (a) receiving text data and medical imaging data, wherein the input text data comprises a segmentation task formulated in natural language with regard to the medical imaging data; (b) generating a text embedding on the basis of the received text data; (c) generating an image embedding on the basis of the received medical imaging data; (d) determining a segmentation of the received medical imaging data on the basis of the generated text embedding and the generated image embedding via a function trained by way of an artificial intelligence algorithm; and (e) outputting the segmentation of the received medical imaging data.

The computer-implemented method according to the second aspect of one or more example embodiments can be carried out in an advantageous manner, for example, with the system according to the first aspect of one or more example embodiments. The features and advantages described herein in relation to the system are therefore also usable for the method and vice versa.

According to a third aspect, one or more example embodiments provides a computer-implemented method for providing a trained function, having the steps: (i) receiving text data and medical imaging data of a first dataset; (ii) receiving segmentation tasks and segmentations of a second dataset, wherein the segmentations correspond to the medical imaging data of the first dataset; (iii) training a function on the basis of the data from the first and second dataset, wherein the training of the function comprises a fine-tuning of the function; and (iv) providing the trained function.

The trained function of the first and second aspect of one or more example embodiments can preferably be trained with the computer-implemented training method according to the third aspect of one or more example embodiments.

The received text data and segmentation masks for the training can originate from clinical reports and/or can be synthetically generated on the basis of keywords relating to at least one lesion type, the size of the lesion, the growth of the lesion and/or the properties of the lesion edges.

Furthermore, the features, advantages or alternative embodiments contained herein can be used in relation to the method according to the third aspect of one or more example embodiments to improve the system according to the first aspect of one or more example embodiments and/or the method according to the second aspect of one or more example embodiments and vice versa.

According to a fourth aspect, one or more example embodiments further provides a computer program product which comprises executable program codes which are configured, on their execution, to carry out the method according to the second aspect and the third aspect of the present invention. It is provided that the program codes for carrying out the second aspect and/or the third aspect of one or more example embodiments can be executed independently of one another.

According to a fifth aspect, one or more example embodiments provides a non-volatile computer-readable data storage medium which comprises executable program codes which are configured, on their execution, to carry out the method according to the second aspect and/or the third aspect of the present invention. It is advantageous if the program codes for carrying out the second aspect and the third aspect of one or more example embodiments can be executed independently of one another.

The non-volatile computer-readable data storage medium can comprise or consist of any type of computer memory store, in particular a semiconductor memory store, such as for example a solid-state memory store. The data storage medium can also comprise or consist of a CD, a DVD, a Blu-Ray disk, a USB memory stick or suchlike.

According to a sixth aspect, one or more example embodiments provides a medical system with a medical scanning system and a system for segmentation of medical imaging data, wherein the medical scanning system is configured to create medical imaging data and wherein the system for segmentation is configured to receive the created imaging data.

A concept underlying one or more example embodiments consists in providing a system for segmentation of medical imaging data, wherein the different segmentation tasks can be defined on the basis of natural language that is processed by a language model. The system creates a text-conditioned segmentation of medical imaging data in that algorithms for segmentation are combined with a language model and are trained together.

The system described above advantageously enables an implementation of a computer-implemented method for segmentation of medical imaging data. Herein, firstly text data and imaging data is received. Thereupon, the text data and imaging data is processed and a corresponding text embedding and image embedding are created. Based thereon, an artificial intelligence algorithm is used which is trained to determine a corresponding segmentation. The segmentation can then be output to an interface in order to offer a visualization of the segmentation.

An advantage of the present invention is that the segmentation tasks are text-conditioned. As distinct from conventional methods which use pre-defined segmentation tasks, one or more example embodiments uses segmentation tasks that are recognized by way of machine learning. The recognition can be improved by training with larger datasets.

A further advantage of one or more example embodiments lies in being able, by way of a segmentation based upon natural language, to transfer refined criteria. Thus it is possible, for example, to create different segmentations, which correspond to different text inputs, from the same imaging data. These changes and adaptations of segmentation masks are initiated by natural language, which leads to a rapid and simple utilization that could be implemented in the medical workflow. Fine details that can be conveyed only with natural language can be taken into account with the present invention.

Advantageous embodiments and developments are disclosed in the further subclaims and in the description, making reference to the figures in the drawing.

According to further embodiments, it is provided that the segmentation task that can be determined with the trained function comprises, for example, the recognition of organs, tissue parts, nerve fibers and lesions. However, one or more example embodiments is not restricted to a list of segmentation tasks, but can also carry out general activities for the recognition of body parts from an imaging method.

According to further embodiments, it is provided that the text encoder unit is configured to map text data in natural language (e.g. ASCII-encoded input) onto a first feature vector; and/or the imaging encoder unit is configured to map medical imaging data onto a second feature vector; and/or the segmentation unit is configured to map the first feature vector and the second feature vector onto a segmentation.

According to further embodiments, it is provided that the text encoder unit is configured to map text data in natural language (e.g. ASCII-encoded input) onto a first feature vector; and that the imaging encoder unit is configured to map medical imaging data onto a second feature vector; and that the segmentation unit is configured to map the first feature vector and the second feature vector onto a segmentation.

According to further embodiments, it is provided that the text encoder unit and/or the imaging encoder unit and/or the segmentation unit are configured as an artificial neural network or are a component of a neural network. The text encoder unit can be based upon a known structure of a transformer network that is known from a large language model (LLM) which comprises a “self-attention” mechanism. Models for the processing of natural language (natural language processing, NLP) such as BERT or SentenceTransformer, can be used. The imaging encoder unit and the segmentation unit can each be a convolutional neural network (CNN).

According to further embodiments, it is provided that the artificial intelligence algorithm comprises a language model, in particular a large language model (LLM), in order to capture semantic information from the text embedding.

A language model or linguistic model or linguistic data processing model can contain a software item that implements one or more algorithms in order, by processing natural language (e.g. in the form of text data), to extract information that is interpreted by a computer and thereby to obtain a meaning therefrom. The language model can be, in particular, a large language model (LLM), i.e. a large generative language model with artificial intelligence, in order to understand, process and generate natural language. Large language models are typically trained with large amounts of data (so-called training at scale) and therefore offer an optimum performance in terms of recognition of natural language.

According to further embodiments, the system further has a storage unit which is configured to store the created segmentation. The storage unit serves as a database for segmentation tasks in which the different segmentations are stored together with the associated text information and imaging data. The storage unit can also serve to provide a tracing capability for the segmentation tasks in conjunction with the imaging data.

According to further embodiments, the trained function comprises a pre-trained linguistic data processing model or a pre-trained image processing model, and wherein the trained function is fine-tuned on the basis of medical reports. The training of the function can take place simultaneously with a text dataset and an imaging dataset. However, it can also be advantageous to use already pre-trained models, for example conventional models of computer vision and linguistic data processing models, in particular if imaging data and text data is difficult to obtain or can be obtained only with a great effort. In order to improve the training, in some embodiments, it is provided to use a combination of pre-trained models together with text data and imaging data. In some embodiments, it is provided that the trained function is subjected to a fine-tuning. Fine-tuning comprises the recording of a model that has been intensively trained for a task in order to refine a second model, so that the second model can fulfill another task. So that the system of one or more example embodiments learns different segmentation tasks, it must be trained with information which contains, in particular: how the lesions, organs or tissue parts can be classified, the relevant features and the best methods for segmentation. This can be achieved by way of a fine-tuning of the large language model (LLM) with the latest clinical guidelines or medical journal articles. The fine-tuning therefore takes place automatically, which leads to a more rapid and efficient training.

According to further embodiments, it is provided that the trained function is based, at least partially, on a neural network, in particular on a transformer model.

A transformer model or transformer network is a special type of machine learning model which is widely used in natural language processing (NLP). It is based upon the “transformers” architecture, which is distinguished by its use of “attention mechanisms”. These mechanisms enable the model to capture the context and the meaning of words in relation to their position in a sentence or text.

A significant feature of the transformer architecture is the use of encoder and decoder units. The encoder processes the input information and converts it into a series of vectors which contain the semantic information of the input. The decoder takes these vectors and creates the final output of the model. Both the encoder and the decoder consist of a plurality of layers, each of which consist of “self-attention” and feed-forward neural networks.

The “self-attention” mechanism is a further central aspect of the transformer architecture. It enables the model to take account of the relationships between different words in a text, regardless of their position. This is particularly useful in the processing of natural language, since the meaning of a word often depends upon its context. By taking account of the context, the model can carry out exact and nuanced predictions and analyses.

Transformer networks are able to recognize complex patterns in data and to learn, which makes them into a powerful tool for many uses in artificial intelligence (AI). Through the combination of encoder and decoder units with self-attention mechanisms, transformer networks can effectively administer a broad range of tasks in the processing of natural language.

A description of the functioning and a possible architecture of transformer networks is given in the document by Ashish Vaswani et al.: “Attention Is All You Need” arXiv: 1706.03762 (2017).

According to further embodiments, the system further comprises a speech recognition interface which is configured to convert speech input into text input and to output the text input to the input unit. The use of such a speech recognition interface has the advantage that the system of one or more example embodiments can be used without technical knowledge. In order to generate a segmentation faster, it can be advantageous to transfer the instructions for the segmentation in speech. This can also be advantageous if one or more example embodiments is utilized in a supporting manner during a surgical intervention.

According to further embodiments, the output interface is configured to recognize the different segmentation tasks which the text data comprises and, for each of the segmentation tasks, to create a separate segmentation of the input imaging data. Above all in a comparison of different criteria, it is advantageous if the different segmentation masks can be seen simultaneously in order to recognize the differences graphically.

According to further embodiments, it is provided that the medical imaging data originates from a computed tomography scan and the segmentation task is oriented to identifying lung nodules. Lung nodules are lesions which occur as small, round or oval tissue lumps in the lung. These lesions can be benign, but can also indicate metastasis.

According to further embodiments, the segmentation task defines which part of lung nodules is to be segmented. For example, segmentation tasks in the context of the identification of lung nodules can be an identification of part-solid lung nodules or an identification of the core of the lung nodules.

Although here and also in the following some functions are described as carried out by units or facilities, this does not necessarily mean that these units and/or facilities are provided as self-contained, mutually associated or mutually separate units and/or facilities. In cases in which one or more units and/or facilities or even a part thereof are provided as software, the units and/or facilities can be implemented by way of program code portions or program code segments that can be separate from one another, but can also be interwoven or integrated with one another. Program codes which have program code portions or program code segments that are distributed over a plurality of units and/or facilities, as is possible, for example, with cloud computing, are also conceivable.

Similarly, in cases where one or more units and/or facilities are provided as hardware, the functions of one or more units and/or facilities can be provided by one and the same hardware component or the functions of a plurality of units and/or facilities can be distributed across a plurality of hardware components which do not necessarily have to correspond to the units and/or facilities. It is therefore to be assumed that each application, each system, each method, etc., which has all the features and functions attributed to a particular unit and/or facilities, comprises or implements this unit and/or these facilities. In particular, it is possible that all the units are implemented by way of program code that is executed by, for example, a server or a cloud computing platform.

The above embodiments and developments can be combined with one another as desired, wherever useful. Further possible configurations, developments and implementations of the invention also include not explicitly mentioned combinations of features described above or in the following in relation to the exemplary embodiments. In particular, a person skilled in the art would also draw upon individual aspects as improvements or enhancements of the respective basic form of the present invention.

The further scope of the applicability of the present method and of the apparatus is apparent from the following figures, the detailed description and the claims. It is however the case that the detailed description and the specific examples, although they give preferred embodiments of the invention, serve primarily for illustration and that various amendments and modifications are discernible within the fundamental concept by persons skilled in the art.

Parts of the different drawings which represent identical or functionally similar elements in the individual views are identified with the same reference signs.

Elements that are shown in the drawings are not necessarily represented to scale. The drawings primarily serve to make the fundamentals and principles of the present invention intelligible and clear. In a similar manner, in some cases, known structures and devices are represented in the form of block diagrams to illustrate possible concepts of the present invention.

The numbering of the steps in the method is intended to simplify their description. They do not necessarily imply a particular sequence of the steps, unless explicitly stated otherwise. In particular, a plurality of steps can also be executed simultaneously and/or multiple times and/or intermediate steps that are not shown can be present.

The exhaustive description of the accompanying drawings contains specific details to enable an extensive understanding of the present invention. It will, however, be clear to a person skilled in the art that the present invention can also be carried out without these specific details.

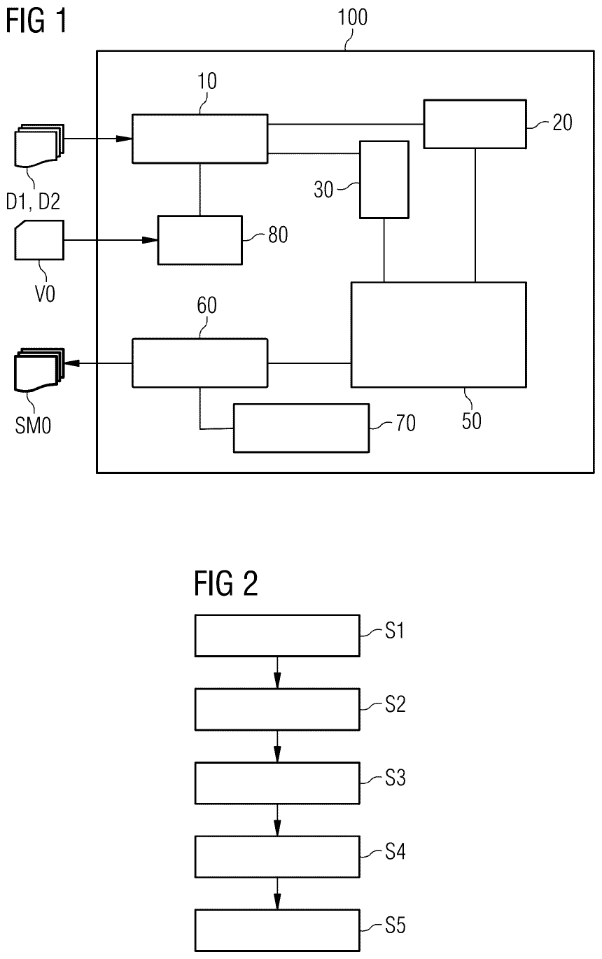

FIG. 1 shows a system 100 for segmentation of medical imaging data according to one embodiment of the invention.

The system 100 comprises an input unit 10, a text encoder unit 20, an imaging encoder unit 30, a segmentation unit 50, an output interface 60, a storage unit 70 and a speech recognition interface 80.

The input unit 10 is configured to receive text data D1 and medical imaging data D2. The imaging data D2 can be transferred from a medical scanning device, such as an X-ray device, a magnetic resonance tomography device or a computed tomography device. The text data comprises one or more segmentation tasks relating to the medical imaging data D2, formulated in natural language. The text data DI can be input, for example, by a physician, another person familiar with these devices or another operating person via a user interface (human-machine interface) that is connected to the input unit 10. The input unit 10 can comprise at least one CPU, a data store and an interface, for example an application programming interface (API). The API can be connected wirelessly or by cable to the medical scanning device and/or the user interface.

The text encoder unit 20 is configured such that it generates a text embedding from the text data DI received from the input unit 10. The text encoder unit 20 is therefore a unit for processing natural language. The text encoder unit 20 can execute a software item, an app or an algorithm with different capabilities for data processing. The text encoder unit 20 can be an artificial neural network (e.g. a transformer network) which is configured to map text data in natural language (e.g. ASCII-encoded input) onto a first feature vector.

The imaging encoder unit 30 is configured to generate an image embedding from the received medical imaging data from the input unit 10. The imaging encoder unit 30 can execute a software item, an app or an algorithm in the specialist field of computer vision. The imaging encoder unit 30 can be an artificial neural network (e.g. a convolutional neural network) which is configured to map medical imaging data onto a second feature vector.

The segmentation unit 50 serves to receive the generated text embedding TE0 and the generated image embedding BE0 and, via a function trained by way of an artificial intelligence algorithm, to determine a segmentation of the received medical imaging data D2. The segmentation comprises at least one segmentation mask. The segmentation is based upon the segmentation task formulated in natural language, which the text data D1 comprises. For example, segmentation tasks in the context of the identification of lung nodules can be an identification of part-solid lung nodules or an identification of the core of the lung nodules.

In preferred embodiments, it is provided that the artificial intelligence algorithm comprises a language model, in particular a large language model (LLM), in order to capture semantic information from the text embedding. Such LLMs can be trained with large amounts of data (so-called training at scale) and therefore provide an optimum performance in terms of recognition of natural language.

The segmentation unit 50 can be an image decoder which is configured to create, from the image embedding and with the aid of the text embedding, an image with the segmentation. The segmentation unit 50 can be an artificial neural network (e.g. a convolutional neural network) which is configured to map the first feature vector and the second feature vector onto a segmentation.

FIG. 1 also represents an output interface 60 which is configured to output the segmentation of the medical imaging data. The output interface 60 can comprise at least one interface, for example an application programming interface (API). The API can be connected wirelessly and/or by cable to a user interface. Furthermore, the output interface 60 can comprise an interface with a display by way of which the segmentation masks can be visualized for an operator and/or user.

The output interface 60 can further be configured to provide more than one segmentation task in relation to the same medical imaging data for visualization. This is advantageous, in particular, in order to make the differences between different segmentation tasks clearly recognizable.

The storage unit 70 is configured to store the created segmentation. The storage unit 70 serves as a database for segmentation tasks in which the different segmentations are stored together with the associated text information and imaging data. From there, the different segmentation tasks can be retrieved and used as needed. The storage unit 70 can also serve to provide a tracing capability for the segmentation tasks in conjunction with the imaging data. The storage unit 70 can be a data storage medium such as, for example, a DVD, a hard disk drive, a USB memory store or suchlike. Alternatively, the storage unit 70 can also be a component part of a computer, a server and/or can be located in a cloud platform.

In some embodiments, as shown in FIG. 1, the system 100 further comprises a speech recognition interface 80. The speech recognition interface 80 is configured to convert language input VO into text input and to output the text input to the input unit 10. The speech recognition interface 80 makes it possible to work faster with the system 100 and offers an alternative to manual actuation of a keyboard. The speech recognition interface 80 can comprise at least one CPU, a data store and an interface, for example an application programming interface (API). The API can be connected wirelessly and/or by cable to the input unit 10.

FIG. 2 shows, in a schematic flow diagram, a representation of the sequence of a computer-implemented method for segmentation of medical imaging data according to one embodiment of the invention. The method can advantageously be carried out with the system 100 of FIG. 1.

In a step S1, text data D1 and medical imaging data D2 is received by an input unit 10. The text data D1 can be entered by a user (e.g. using a suitable user interface). The text data defines a segmentation task. Imaging data D2 can be transferred from a medical scanning device, such as an X-ray device or a computed tomography device.

In a step S2, a text embedding is generated. The text embedding corresponds to a representation of the received text data D1 that can be used for a post-processing by a language model.

In a further step S3, an image embedding is generated on the basis of the received medical imaging data. The image embedding corresponds to a representation of the received imaging data D2 that can be post-processed by way of a computer vision software.

In a step S4, on the basis of the generated text embedding and the generated image embedding, a segmentation of the received medical imaging data D2 is determined. This determination takes place by way of the use of an artificial intelligence algorithm which is preferably based upon a transformer model. The artificial intelligence algorithm is trained to determine a segmentation from the image embedding and the text embedding. This segmentation comprises at least one segmentation mask.

In a step S5, the segmentation is output. This can take place by way of a corresponding interface, so that the segmentation mask which corresponds to the segmentation task can be visualized. The segmentation that is output can further comprise text details which place the segmentation mask and the segmentation task in relation to one another.

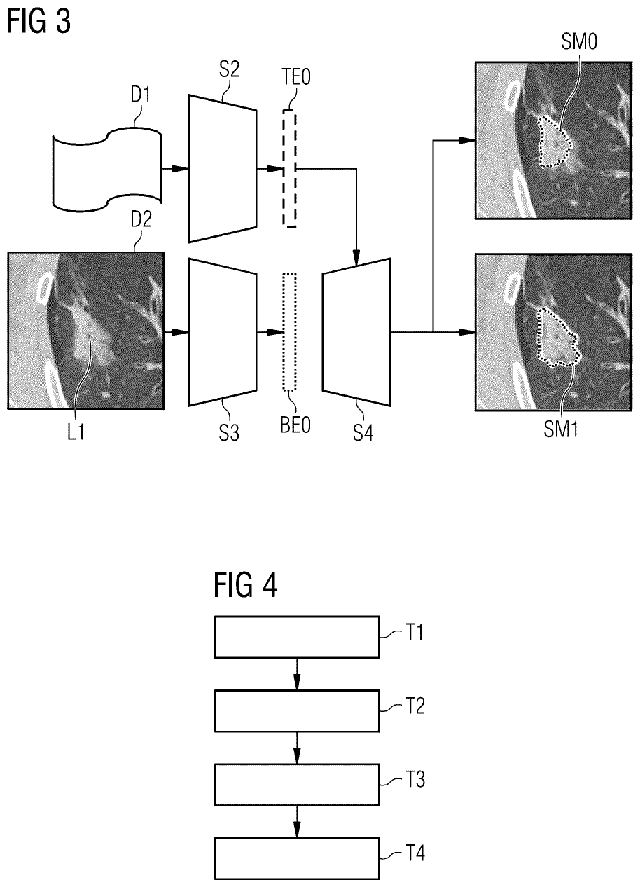

FIG. 3 shows, for example, a schematic flow diagram to illustrate the sequence of a computer-implemented method for the segmentation of medical imaging data according to one embodiment of the invention.

Text data D1 and imaging data D2 of a region with a lung nodule L1 are received. On the basis of the text data D1 (e.g. “Identification of a part-solid lung nodule”), a text embedding TE0 is generated (identified as step S2). In parallel therewith, an image embedding BE0 is generated on the basis of the imaging data D2 (identified as step S3). The image embedding BE0 and the text embedding TE0 are processed by an artificial intelligence algorithm and a segmentation is determined on the basis of this processing (step S4). On the basis of the segmentation, a segmentation mask SM0 is generated which represents the edges of a part-solid lung nodule.

In FIG. 3, a second segmentation mask SM1 is also shown. The second segmentation mask SM1 can be initiated by a new text input (not shown). On the basis of the new text input (e.g. “Identification of the solid core of the lung nodule”), in a step S2, a new text embedding (not shown) is generated. A corresponding segmentation is determined and based thereupon, the segmentation mask SM1 is created. According to one or more example embodiments, the image embedding BE0 during the processing of the segmentation mask SM1 requires no change or post-processing. By way of this procedure and, in particular, with the same image embedding BE0, for example, the segmentation mask SM0 could identify the edges of a part-solid lung nodule and the segmentation mask SM1 could identify the solid core of the lung nodule.

FIG. 4 shows, in a schematic flow diagram, an illustration of the sequence of a computer-implemented method for training a function for the determination of different segmentation tasks according to one embodiment of the invention. The method can advantageously be carried out with the system 100 of FIG. 1.

In a step T1, text data and medical imaging data is received from a first dataset. The received text data and segmentation masks could be generated in an automated manner from clinical reports and/or could be synthetically generated, on the basis of keywords relating to at least one lesion type, the size of the lesion, the growth of the lesion and/or the properties of the lesion edges.

In a further step T2, segmentation tasks and segmentation masks of a second dataset are received, wherein the segmentation masks correspond to the medical imaging data of the first dataset. The segmentation masks are provided with annotations on the basis of previously provided images or reports.

In a step T3, a function is trained on the basis of the data of the received first and second dataset. In some embodiments, the function can comprise a pre-trained language model, e.g. a generative pre-trained transformer (GPT) language model or a pre-trained image processing model, e.g. SimCLR or SwAV. The function can also comprise a language model and an image processing model jointly pre-trained with “contrastive language-image pre-training” (CLIP). Databases such as ImageNet COCO, GLUE, SQUAD or PubMed can be used to generate training datasets. In this way, the performance of the function can be optimized.

In some embodiments, it is provided that the trained function is subjected to a fine-tuning. Information comprising: a) how the lesions, organs or tissue parts can be classified; b) the relevant features; and c) the best methods for the segmentation can advantageously be drawn from the latest clinical guidelines or medical journal articles. The fine-tuning therefore takes place automatically, which leads to a fast and efficient training.

In a step T4, the trained function is provided. This trained function can be used for carrying out the computer-implemented method for segmentation of medical imaging data according to the second aspect of one or more example embodiments.

FIG. 5 shows a representation of different segmentation masks in association with the identification of a lung nodule L1 according to one embodiment of the invention. The imaging data received by the system 100 is arranged in the first row and has been recorded during a computed tomography scan. It represents (from left to right) the axial, coronal and sagittal view of a lung nodule. Shown in FIG. 5 are two segmentation masks SM0, SM1. The first segmentation mask SM0 can be created in association with a text input (e.g. “Identification of a part-solid lung nodule”). The second segmentation mask SM1 can be created in association with a text input (e.g. “Identification of the solid core of the lung nodule”). According to one or more example embodiments, the system 100 can therefore be prompted with natural language to generate the corresponding segmentation masks SM0, SM1. The system 100 can preferably also represent the segmentation masks SM0, SM1 simultaneously so that the differences are clearly apparent.

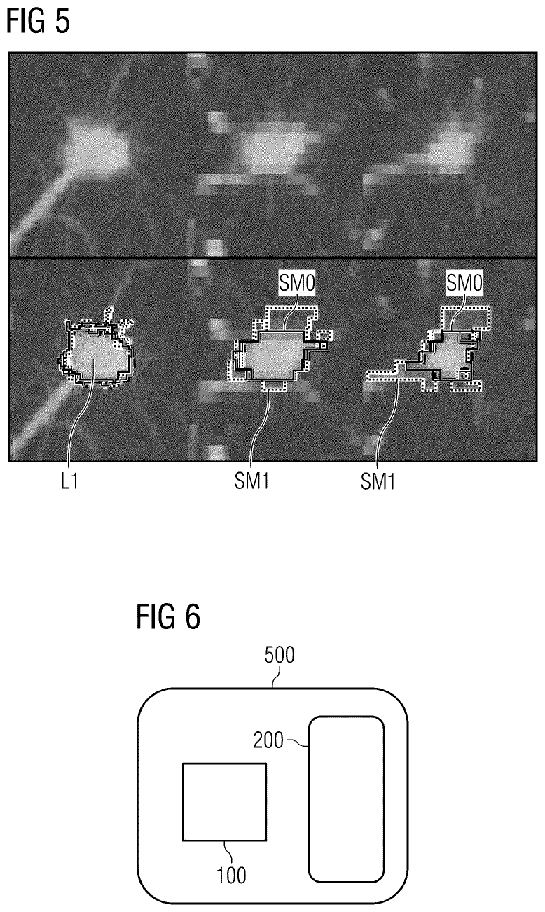

FIG. 6 shows a schematic block diagram which represents a medical system 500 with a medical scanning system 200 and a system 100 for segmentation of medical imaging data according to one embodiment of the sixth aspect of the present invention.

The medical scanning system 200 is configured to generate imaging data. The medical scanning system 200 can be, in particular, a device which can carry out an imaging method in the field of medical diagnostics. The device can be, for example, a magnetic resonance tomography device, an X-ray device or a computed tomography device.

The system 100 for segmentation of medical imaging data is a system 100 according to the first aspect of one or more example embodiments as shown, for example, in FIG. 1. The system 100 is configured to record the imaging data generated by the medical scanning system 200. The system 100 can be implemented on the medical scanning system 200. Additionally or alternatively, it can also be connected thereto wirelessly or by cable.

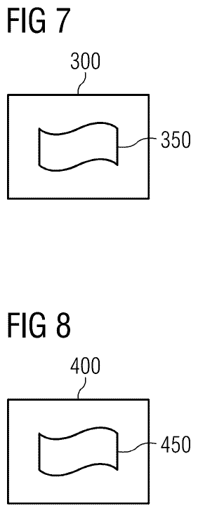

FIG. 7 shows a schematic block diagram which illustrates a computer program product 300 according to one embodiment of the third aspect of the present invention. The computer program product 300 comprises executable program codes 350 which are configured such that, when executed, they carry out the method according to any embodiment of the second aspect and/or of the third aspect of the present invention, in particular as described above in relation to the preceding figures. The program codes for carrying out the second aspect and/or the third aspect of one or more example embodiments can be carried out independently of one another.

FIG. 8 shows a schematic block diagram which illustrates a non-volatile computer-readable data storage medium 400 according to one embodiment of the fourth aspect of the present invention. The data storage medium 400 comprises executable program codes 450 which are configured such that, when executed, they carry out the method according to any embodiment of the second aspect and/or of the third aspect of the present invention, in particular as described above in relation to the preceding figures. The program codes for carrying out the second aspect and/or the third aspect of one or more example embodiments can be carried out independently of one another.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that encompasses relationship a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SOC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A system configured to segment medical imaging data, comprising:
an input unit configured to receive text data and the medical imaging data, wherein the received text data comprises a segmentation task formulated in natural language with regard to the medical imaging data;
a text encoder unit configured to generate a text embedding based on the received text data;
an imaging encoder unit configured to generate an image embedding based on the received medical imaging data;
a segmentation unit configured to receive the generated text embedding and the generated image embedding and to determine a segmentation of the received medical imaging data via a function trained by an artificial intelligence algorithm; and
an output interface configured to output the segmentation of the medical imaging data.

2. The system of claim 1, wherein the segmentation unit is configured to recognize organs, tissue parts, nerve fibers and lesions.

3. The system of claim 1, wherein at least one of:
the text encoder unit is configured to map text data in natural language onto a first feature vector;
the imaging encoder unit is configured to map medical imaging data onto a second feature vector; or
the segmentation unit is configured to map the first feature vector and the second feature vector onto a segmentation.

4. The system of claim 1, wherein at least one of the text encoder unit, the imaging encoder unit, or the segmentation unit is configured as an artificial neural network or is a component of a neural network.

5. The system of claim 1, wherein the artificial intelligence algorithm comprises a language model to capture semantic information from the text embedding.

6. The system of claim 1, further comprising a storage unit configured to store the created segmentation.

7. The system of claim 1, wherein
the trained function comprises a pre-trained linguistic data processing model or a pre-trained image processing model, and
the trained function is fine-tuned based on medical reports.

8. The system of claim 1, wherein the trained function is based on a neural network.

9. The system of claim 1, further comprising:
a speech recognition interface configured to convert speech input into text input and to output the converted text input to the input unit.

10. The system of claim 1, wherein the output interface is further configured to recognize different segmentation tasks which the text data comprises and for each of the segmentation tasks, to create a separate segmentation of the received imaging data.

11. The system of claim 1, wherein the medical imaging data originates from a computed tomography scan and the segmentation task is oriented to an identification of lung nodules.

12. The system of claim 11, wherein the segmentation task describes a part of the lung nodules to be segmented.

13. A computer-implemented method for segmentation of medical imaging data, the method comprising:
receiving text data and medical imaging data, the received text data including a segmentation task formulated in natural language with regard to the medical imaging data;
generating a text embedding based on the received text data;
generating an image embedding based on the received medical imaging data;
determining a segmentation of the received medical imaging data based on the generated text embedding and the generated image embedding via a function trained by an artificial intelligence algorithm; and
outputting the segmentation of the received medical imaging data.

14. A computer-implemented method for providing a trained function, the method comprising:
receiving text data and medical imaging data of a first dataset;
receiving segmentation tasks and segmentation masks of a second dataset, wherein the segmentation masks correspond to the medical imaging data of the first dataset;
training a function based on the received first dataset and the second dataset, the training of the function including fine-tuning of the function; and
providing the trained function.

15. The computer-implemented method of claim 14, wherein the received text data and segmentation masks have been at least one of generated automatically from clinical reports or are synthetically generated based on keywords relating to at least one of at least one lesion type, a size of a lesion, a growth of the lesion or properties of lesion edges.

16. A medical system comprising:
a medical scanning system configured to generate medical imaging data; and
a system configured to segment the medical imaging data, wherein the system is configured to receive the generated imaging data.

17. A non-transitory computer program product comprising executable program codes, when executed by a system, cause the system to perform the method of claim 13.

18. A non-volatile computer-readable data storage medium comprising executable program codes, when executed by a system, cause the system to perform the method of claim 13.

19. The system of claim 5, wherein the artificial intelligence algorithm comprises a large language model.

20. The system of claim 8, wherein the trained function is based on a transformer model.
